(19) United States
(12) Patent Application Publication
(10) Pub. No.: US 2023/0201112 A1
(43) Pub. Date: Jun. 29, 2023

(54) DISSOLVABLE MEDICAL DEVICE AND KIT FOR CORNEAL SURFACE PROTECTION

(71) Applicant: Alcon Inc., Fribourg (CH)

(72) Inventors: Howard Allen Ketelson, Dallas, TX (US); Rekha Rangarajan, Fort Worth, TX (US); Patrick John Sadd, Colleyville, TX (US)

(21) Appl. No.: 18/145,256

(22) Filed: Dec. 22, 2022

Related U.S. Application Data
(60) Provisional application No. 63/265,968, filed on Dec. 23, 2021.

(51) Int. Cl. A61K 9/00; A61K 9/70; A61K 47/36; A61K 47/32; A61K 47/10

(52) U.S. Cl. CPC A61K 9/0051; A61K 9/70; A61K 47/36; A61K 47/32; A61K 47/10

(57) ABSTRACT

A dissolvable medical device for protecting a corneal surface and providing lubrication and hydration to the corneal surface during ophthalmic surgery comprising: a polymeric film has sufficient dimensions to substantially cover a cornea when applied to an eye, wherein the polymeric film comprising one or more mucoadhesive polymers. The polymeric film dissolves between 15 minutes to 120 minutes to release the mucoadhesive polymers after applying the polymeric film to the eye and the polymeric film provides more effective corneal surface protection relative to a saline treatment based on Mean Green Fluorescence Index test (demonstrates corneal damage) during a simulated surgery. In addition, a kit for use in ophthalmic surgical procedures comprises 1) the at least one dissolvable medical device and 2) at least one viscosurgical viscoelastic or at least one ophthalmoscopic surgical contact lens.

### FIELD OF THE DISCLOSURE

The present disclosure generally relates to a dissolvable medical device and a kit for placement on the outer surface (Cornea) of the eye to protect a corneal surface and providing lubricant and hydration to the corneal during eye surgery while also serving as a stabilizing base for an ophthalmoscopic surgical contact lens.

### BACKGROUND

There are several known viscous or viscoelastic agents for ophthalmic surgical use. They are used by the skilled ophthalmic surgeon for several surgical purposes, including maintenance of intraocular spaces, protection of ophthalmic tissues, particularly corneal endothelial cells, and as an aid in manipulating ophthalmic tissues. To decrease postoperative endothelial cell loss, different ocular viscoelastic devices (OVDs) have been proposed to facilitate surgical maneuvers, maintain space during surgery, and protect the endothelial cells. There are four broad categories of viscoelastic devices (OVDs): 1) dispersives, 2) cohesives, 3) combination agents and 4) visco-adaptives. Dispersive OVDs, with a lower viscosity, have the ability to coat intraocular structures, and they tend to stay in place during the fluidics of phacoemulsification surgery. They are good for partitioning spaces within the eye, such as after a rupture of the posterior capsule. Due to this retentiveness, removal of dispersive OVDs requires more effort at the end of surgery. Cohesive OVDs, with a higher viscosity, are able to pressurize the eye and create space, such as during IOL insertion. Because they are more viscous and more solid in their behavior, they are ideal for flattening the anterior capsule to facilitate capsulorrhexis creation or for deepening a shallow anterior chamber. Due to their cohesiveness, the entire mass of viscoelastic tends to stick together. This makes removal at the end of the case easy, however it makes chamber retention less effective. Protecting the corneal endothelium throughout the surgery is a dispersive characteristic, while flattening the anterior lens capsule during capsulorrhexis is a cohesive attribute. DiscoVisc (Alcon) is an example of single-syringe agent having characteristics of both higher viscosity cohesives as well as lower viscosity dispersives, these agents can be thought of as highly viscous dispersive OVDs. Another approach is using two separated OVDs offering more versatility than a single agent, for example, Alcon's DuoVisc system consists of Viscoat (dispersive) and ProVisc (cohesive). A visco-adaptive OVD behaves as a super-cohesive viscoelastic to pressurize and create space yet can provide the protection of dispersives. It takes advantage of both properties of viscoelastics, by being a substance that changes its behavior at different flow rates. The lower the flow rate, the more viscous and cohesive the OVD becomes. The higher the flow rate, the more pseudodispersive the viscoelastic is allowing for better protection of the corneal endothelial cells from injury during the phacoemulsification step.

From the above discussion, viscoelastic devices (OVDs) provide good protection of corneal endothelial cells during eye surgery. There is still a need for a medical device that can provide good protection of corneal epithelium during a surgical procedure. Currently, surgeons keep the exposed ocular surface moist by treating the cornea with a Balanced Salt Solution or saline flush every couple of minutes during the entire surgical procedure.

There are challenges when utilizing an ophthalmoscopic surgical contact lens during ophthalmoscopic surgery. They require use of a viscoelastic to provide a bed to set the ophthalmoscopic surgical contact lens on and they require constant use of BSS to clear the lens to keep a consistent crisp view. It is often difficult to find a technician that can hold or position the ophthalmoscopic surgical contact lens and keep it stable. Those ophthalmoscopic surgical contact lens lenses that are “non-handled” and set on top of the cornea without a technician holding the lens but tend to move or slide when a surgeon is operating and scleral depressing. These ophthalmoscopic surgical contact lens lenses can also cause corneal abrasions if not handled properly.

There is a need for a medical device that can provide good protection of corneal epithelium during a surgical procedure. Currently, surgeons keep the exposed ocular surface moist by treating the cornea with a Balanced Salt Solution or saline flush every couple of minutes during the entire surgical procedure. There is also a need for a sustained delivery device that can be applied to the corneal surface that can provide hydration and protection of the ocular surface during surgery and can be used to anchor a lens to the eye during macular surgery that would improve stability and improve view. Furthermore, there is still a need to have a kit including the devices to protect corneal epithelium and to work as a protective lens anchor when utilizing an ophthalmoscopic surgical contact lens.

### SUMMARY

The invention provides a dissolvable medical device for protecting a corneal surface and providing lubrication and hydration to the corneal surface during ophthalmic surgery comprising: a polymeric film has sufficient dimensions to substantially cover a cornea when applied to an eye, wherein the polymeric film comprising one or more mucoadhesive polymers, wherein the polymeric film dissolves between 15 minutes to 120 minutes to release the mucoadhesive polymers after applying the polymeric film to the eye, wherein the polymeric film provides more effective corneal surface protection relative to a saline treatment based on Mean Green Fluorescence Index test (demonstrates corneal damage) during a simulated surgery.

The invention also provides a kit for use in ophthalmic surgical procedures comprising: 1) at least one dissolvable medical device for protecting a corneal surface and providing lubrication and hydration to the cornea before and during an ophthalmic surgery comprising: a polymeric film has sufficient dimensions to substantially cover a cornea when applied to an eye, wherein the polymeric film comprising one or more mucoadhesive polymers, wherein the polymeric film dissolves between 15 minutes to 120 minutes to release the mucoadhesive polymer after applying the polymeric film to the eye, wherein the polymeric film provides more effective corneal surface protection relative to a saline treatment based on Mean Green Fluorescence Index test (demonstrates corneal damage) during a simulated surgery, and 2) at least one viscosurgical viscoelastic or at least one ophthalmoscopic surgical contact lens, wherein the at least one viscosurgical viscoelastic comprising a cohesive viscoelastic, wherein cohesive viscoelastic is a hyaluronate-based viscoelastic, wherein the at least one ophthalmoscopic surgical contact lens comprising: an optic including an anterior surface having an aspheric base profile and a posterior surface having a shape substantially corresponding to a shape of a cornea of an eye; and a rim comprising a cylindrical tube that circumferentially surrounds the optic and extend above the anterior surface of the optic, wherein the kit provides protection for both corneal endothelium and corneal epithelium or stabilization of the ophthalmoscopic surgical contact lens.

The invention, in another aspect, provides a method for conducting ophthalmic surgery in a human eye having a cornea, an anterior chamber, a posterior chamber and a capsular bag located within the posterior chamber, comprising:

placing a dissolvable medical device for protecting a corneal surface and providing lubricant and hydration to the corneal during ophthalmic surgery, wherein the dissolvable medical device comprising: a polymeric film has sufficient dimensions to substantially cover a cornea when applied to an eye, wherein the polymeric film comprising one or more mucoadhesive polymers, wherein the polymeric film dissolves between 15 minutes to 120 minutes to release the mucoadhesive polymer after applying the polymeric film to the eye, wherein the polymeric film provides more effective corneal surface protection relative to a saline treatment based on Mean Green Fluorescence Index test (demonstrates corneal damage) during a simulated surgery,

surgically opening the human eye, wherein surgically opening is selected from a group consisting of incising, slicing, and injecting the corneal surface.

### BRIEF DESCRIPTION OF THE DRAWINGS

For a more complete understanding of this disclosure, reference is now made to the following description, taken in conjunction with the accompanying drawings, in which:

FIG. 1 illustrates Corneal surface protection in mice treated with the dissolvable medical device (shield) vs. saline (PBS) during a simulated surgery.

FIGS. 2A-2C illustrate the compatibility of the polymeric film during simulated surgical procedures.

FIGS. 3A-3B illustrate the compatibility of the polymeric film on visualization of the posterior chamber i.e., retina during a simulated surgery.

FIG. 4 illustrates an ophthalmoscopic surgical contact lens.

### DETAILED DESCRIPTION

Reference now will be made in detail to the embodiments of the invention, one or more examples of which are set forth below. Each example is provided by way of explanation of the invention, and it is not a limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment. Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only and is not intended as limiting the broader aspects of the present invention.

Unless defined otherwise, all technical and scientific terms used herein have the same meaning as commonly understood by one of ordinary skill in the art to which this invention belongs. Generally, the nomenclature used herein and the laboratory procedures are well known and commonly employed in the art. Conventional methods are used for these procedures, such as those provided in the art and various general references. Where a term is provided in the singular, the inventors also contemplate the plural of that term. As employed throughout the disclosure, the following terms, unless otherwise indicated, shall be understood to have the following meanings.

The invention, in one aspect, provides a dissolvable medical device for protecting a corneal surface and providing lubricant and hydration to the corneal during ophthalmic surgery comprising: a polymeric film has sufficient dimensions to substantially cover a cornea when applied to an eye, wherein the polymeric film comprising one or more mucoadhesive polymers, wherein the polymeric film dissolves between 15 minutes to 120 minutes to release the mucoadhesive polymers after applying the polymeric film to the eye, wherein the polymeric film provides more effective corneal surface protection relative to a saline treatment based on Mean Green Fluorescence Index test (demonstrates corneal damage) during a simulated surgery.

It has been discovered that a dissolvable medical device comprising a polymeric film which comprises one or more mucoadhesive polymers is well suitable for protecting corneal surface and providing lubricant and hydration to the corneal during an ophthalmic surgery. The polymeric film of the invention is characterized by having a dimension substantially covering a cornea when applied to an eye and characterized by dissolving between 15 minutes and 120 minutes to release the mucoadhesive polymers after the film after applying to the eye. In addition, the dissolved polymeric film is not impeding visualization during maintaining on outer surface of the eye.

The dissolvable medical device as an ophthalmic surgical device can offer some advantages over a collagen shield, which is typically used post-operatively. First, it is much easier to dissolve the polymeric film of the present invention than to dissolve a collagen shield to enable use during surgery. The currently commercially available collagen shields have dissolution times of 12, 24, and 72 hours. The amount of crosslinking induced in the collagen shield by UV irradiation during manufacture determines the length of time the shield will remain intact and on the eye. In contrast, the polymeric film of the present invention has dissolution times of between 15 minutes to 120 minutes, which is advantageous for use as a corneal surface protectant during surgery. Second, the dissolved polymeric film of the present invention is not impeding visualization when present on the ocular surface of the eye. In contrast, upon contact with enzymes that are present in the tears on the eye, the collagen shield will begin to swell and become cloudy, resulting in a loss of transparency. The loss of transparency of the collagen shields shortly after being placed on the eye is the biggest problem with the collagen shields. Because the cloudiness interferes during the possible followed surgical procedures and impeded visualization during surgical procedures. Collagen shields are used post-operatively only to promote ocular surface healing. The lubrication/protection property is minimal.

Furthermore, the polymeric film of the present invention provides more effective corneal surface protection relative to a saline treatment based on Mean Green Fluorescence Index test (demonstrates corneal damage) during a simulated surgery.

The biomaterial for forming a dissolvable medical device according to embodiments of the present disclosure may be comprised of one or more polymers that are biocompatible with the ocular surface and tear film. Polymers that may be used in dissolvable medical device according to embodiments of the present disclosure include, but are not limited to, hyaluronic acid (in acid or salt form), hydroxypropylmethylcellulose (HPMC), methylcellulose, tamarind seed polysaccharide (TSP), Galactomannans, for examples; guar and derivatives thereof such as hydroxypropyl guar (HP guar), scleroglucan poloxamer, poly(galacturonic) acid, sodium alginate, pectin, xanthan gum, xyloglucan gum, chitosan, sodium carboxymethylcellulose, polyvinyl alcohol, polyvinyl pyrrolidine, carbomer, polyacrylic acid and/or combinations thereof.

The preferred biocompatible polymers are hyaluronic acid, guar, and derivatives and/or combinations thereof. Hyaluronic acid is an unsulphated glycosaminoglycan composed of repeating disaccharide units of N-acetylglucosamine (GlcNAc) and glucuronic acid (GlcUA) linked together by alternating beta-1,4 andbeta-1,3 glycosidic bonds. Hyaluronic acid is also known as hyaluronan, hyaluronate, or HA. As used herein, the term hyaluronic acid also includes salt forms of hyaluronic acid such as sodium hyaluronate. A preferred hyaluronic acid is sodium hyaluronate. The weight average molecular weight of the hyaluronic acid used in insert of the present invention may vary but is typically weight average of 0.75 to 5.0 M Daltons. In one embodiment, the HA has a weight average molecular weight of 0.75 to 4 M Daltons. In another embodiment, the HA has a weight average molecular weight of 1 to 4 M Daltons.

The galactomannans of the present invention may be obtained from numerous sources. Such sources include from fenugreek gum, guar gum, locust bean gum and tara gum. Additionally, the galactomannans may also be obtained by classical synthetic routes or may be obtained by chemical modification of naturally occurring galactomannans. As used herein, the term “galactomannan” refers to polysaccharides derived from the above natural gums or similar natural or synthetic gums containing mannose or galactose moieties, or both groups, as the main structural components. Preferred galactomannans of the present invention are made up of linear chains of (1-4)-.beta.-D-mannopyranosyl units with. Alpha.-D-galactopyranosyl units attached by (1-6) linkages. With the preferred galactomannans, the ratio of D-galactose to D-mannose varies, but generally will be from about 1:2 to 1:4. Galactomannans having a D-galactose:D-mannose ratio of about 1:2 is most preferred. Additionally, other chemically modified variations of the polysaccharides are also included in the “galactomannan” definition. For example, hydroxyethyl, hydroxypropyl and carboxymethylhydroxypropyl substitutions may be made to the galactomannans of the present invention. Non-ionic variations to the galactomannans, such as those containing alkoxy and alkyl (C1-C6) groups are particularly preferred when a soft gel is desired (e.g., hydroxylpropyl substitutions). Substitutions in the non-cis hydroxyl positions are most preferred. An example of non-ionic substitution of a galactomannan of the present invention is hydroxypropyl guar, with a molar substitution of about 0.4. Anionic substitutions may also be made to the galactomannans. Anionic substitution is particularly preferred when strongly responsive gels are desired, Preferred galactomannans of the present invention are guar and hydroxypropyl guar. Hydroxypropyl guar is particularly preferred. The weight average molecular weight of the Hydroxypropyl guar in the dissolvable medical device of the present invention may vary but is typically but is typically 1 to 5M Daltons. In one embodiment, the Hydroxypropyl guar has a weight average molecular weight of 2 to 4M Daltons. In another embodiment, the Hydroxypropyl guar has a weight average molecular weight of 3 to 4 M Daltons.

Polymers used in dissolvable medical devices according to embodiments of the present disclosure should be non-toxic and able to solubilize in eye fluids to ensure that the insert is eventually cleared from the eye, generally within 15 to 120-minute time frame. It should be appreciated that the polymer(s) selected should be mucoadhesive. It also should be appreciated that one or more polymers may be blended according to embodiments of the present disclosure. For example, in an embodiment of the present disclosure, hyaluronic acid (HA) may be blended with tamarind seed polysaccharide (TSP) because TSP has been shown to increase residence time of HA in aggregate blends and the blend has desired film mechanical and lubrication properties. In other embodiments of the present disclosure, as described in further detail below, hyaluronic acid may be combined with HP guar.

In some embodiments of the present disclosure, the preferred biocompatible polymers also include polyvinyl pyrrolidine (PVP). PVP is also a mucoadhesive polymer. The weight average molecular weight of the PVP in the polymeric film of the present invention may vary but is typically 4,000 Dalton to 3 M Daltons. In one embodiment, the PVP has a weight average molecular weight of 40 K Daltons to 2 M Daltons. In another embodiment, the PVP has a weight average molecular weight of 0.5 M Daltons to 2 M Daltons.

In some embodiments of the present disclosure, a softener and/or plasticizer may be added to the one or more polymers to facilitate fabrication of a softer, malleable delivery system and provide improved comfort in covering the cornea. A plasticizer may soften the material to provide for desirable dissolution rates. It should be appreciated softeners and/or plasticizers may be low or high-molecular weight compounds, including not limited to, polyethylene glycol (PEG) and derivatives thereof, water, Vitamin E, and triethyl citrate. The weight average molecular weight of the PEG in the polymeric film of the present invention may vary but is typically 200 Dalton to 100,000 Daltons. In one embodiment, the PEG has a weight average molecular weight of 200 to 12000 Daltons. In another embodiment, the PEG has a weight average molecular weight of 200 to 6000 Daltons.

In some embodiments, the HP guar is present in an amount of from about 5% to about 60% w/w, preferably 15% to about 50% w/w, more preferably 25% to about 40 w/w by dry weight of the polymeric film. The PVP is present in an amount of from about 1% to about 30% w/w, preferably 5% to about 25% w/w, more preferably 10% to about 20 w/w by dry weight of the polymeric film. The hyaluronic acid (HA) is present in an amount of from about 5% to about 60% w/w, preferably 15% to about 50% w/w, more preferably 25% to about 40 w/w by dry weight of the polymeric film. The PEG is present in an amount of from about 1% to about 30% w/w, preferably 5% to about 25% w/w, more preferably 10% to about 20 w/w by dry weight of the polymeric film. According to the present application, the total amount of ingredients of the polymeric dissolvable medical devices is equal to 100% w/w.

The overall dry weight or mass of the polymeric film may be in the range of about 1 to about 12 mg, or about 2 to about 10 mg, and in particular embodiments may be from about 3 to about 9 mg.

In some embodiments, the polymeric film has a thickness of about 50-300 μm, about 120-250 μm, about 140-200 μm, or preferably about 120 μm.

In some embodiments, the polymeric film has circular shape about 2 mm to 13 mm in diameter or other shapes have the same area corresponding to circular shape about 2 mm to 13 mm in diameter. In still some embodiments, the polymeric film has a contact lens shape and prefers about 11 mm to 13 mm in diameter.

The invention, in another aspect, provides a kit for use in ophthalmic surgical procedures comprising: 1) at least one dissolvable medical device for protecting a corneal surface and providing lubrication and hydration to the corneal before ophthalmic surgery comprising: a polymeric film has sufficient dimensions to substantially cover a cornea when applied to an eye, wherein the polymeric film comprising one or more mucoadhesive polymers, wherein the polymeric film dissolves between 15 minutes to 120 minutes to release the mucoadhesive polymers after applying the polymeric film to the eye, wherein the polymeric film provides more effective corneal surface protection relative to a saline treatment based on Mean Green Fluorescence Index test (demonstrates corneal damage) during a simulated surgery, and 2) at least one viscosurgical viscoelastic or at least one ophthalmoscopic contact lens, wherein the at least one viscosurgical viscoelastic comprising a cohesive viscoelastic, wherein cohesive viscoelastic is a hyaluronate-based viscoelastic, wherein the at least one ophthalmoscopic surgical contact lens comprising: an optic including an anterior surface having an aspheric base profile and a posterior surface having a shape substantially corresponding to a shape of a cornea of an eye; and a rim comprising a cylindrical tube that circumferentially surrounds the optic and extend above the anterior surface of the optic, wherein the kit provides protection for both corneal endothelium and corneal epithelium or stabilization of the ophthalmoscopic surgical contact lens.

According to the present disclosure, two viscoelastic agents may be used during cataract surgery was performed by a skilled ophthalmic surgeon. One agent is used during capsulotomy and irrigation/aspiration or phacoemulsification of the cataractous lens (Stage 1) and a different agent is used following extraction of the lens and during implantation of an intraocular lens (Stage 2). The agent used during Stage 1 of the surgery should be adherent enough to be retained in the anterior chamber, that is, it should effectively maintain anterior chamber space and relieve lens convexity, i.e., flatten the lens somewhat so that a capsulotomy can be done with more control and less chance of peripheral capsular tearing. The agent should also protect the tissues, particularly the corneal endothelial cells, from trauma resulting from shear forces and direct contact from nuclear fragments and instruments. The agent used during Stage 2 should effectively allow for implantation of an IOL by being used to manipulate tissue, i.e., filling and opening the capsular bag which is where the IOL will be placed and maintaining the anterior chamber prior to and during implantation of the IOL.

For Stage 1 of a cataract procedure, it is appropriate to employ an agent that has characteristics that will enable it to function as previously discussed, i.e., it will maintain the anterior chamber and protect the ophthalmic tissues from trauma during capsulotomy and removal of the cataract. The agent to be used during Stage 2 should have characteristics that allow it to be used as a tool for manipulating tissue, i.e., inflation of the capsular bag and insertion of an IOL within the bag. It should also be relatively easy to remove from the eye after IOL implantation.

Viscoelastic agents which are useful for methods of the present invention include but are not limited to: sodium hyaluronate, chondroitin sulfate, polyacrylamide, HPMC, proteoglycans, collagen, methylcellulose, carboxymethyl cellulose, ethylcellulose, and keratin of various molecular weights, or combinations thereof. Whether it is appropriate to use an agent for Stage 1 or Stage 2 of a cataract procedure will depend on the physical and chemical characteristics of each agent or combination, including, but not limited to, their molecular weight, viscosity, pseudoplasticity, elasticity, rigidity, coatability, cohesiveness, and molecular charge, and the agent's concentration in a product.

The preferred method involves the use of an agent containing sodium hyaluronate and chondroitin sulfate, such as Viscoat®, during Stage 1 of the procedure and a relatively high molecular weight sodium hyaluronate product, such as Provisc® or Healon® during Stage 2. More particularly, Viscoat®is used upon the surgeons' entrance into the anterior chamber mainly to fill and maintain the chamber and protect the tissues during capsulotomy and phacoemulsification and or irrigation/aspiration and removal of the cataractous lens elements. A high molecular weight sodium hyaluronate product, such as Provisc® or Healon® is then introduced as a viscoirrigant replacing the Viscoat® or after removal of some or all the Viscoat®. It can also be used to maintain the anterior chamber but is introduced into the empty capsular bag to inflate it for introduction and placement of an IOL. Upon completion of IOL placement the sodium hyaluronate can be removed to help prevent a post-surgical sharp increase in intraocular pressure. The use of these two agents during cataract surgery provides for optimal maintenance of the anterior chamber, protection of tissues, and manipulation of the capsular bag for IOL implantation.

More than one agent can be employed by the skilled surgeon in a variety of procedures by choosing an agent with the desired characteristics to either help manipulate tissues or function as an adhesive, protective agent. For example, a vitrectomy may require much tissue manipulation and therefore, a pure sodium hyaluronate product would be most useful as an aid to such manipulation. If the procedure involves a detached retina, use of a product with good adhesive properties, like Viscoat® can be employed prior to closing to serve as a tamponade.

According to the present application, the ophthalmoscopic surgical contact lens includes an optic surrounded by a rim and at least one flange. The optic includes an aspheric anterior surface and a posterior surface having a shape substantially corresponding to the shape of a human cornea. The rim, comprising an edge surrounding the optic, provides the user with a gripping surface conducive to manual positioning and repositioning of the lens against a human eye. The flange may include a plurality of tabs extending from a periphery of the flange, wherein each tab is shaped and configured to conform to the curvature of a human sclera.

According to the present application, the dissolvable medical device improves the visualization of structures within the interior of an eye, such as may be necessary during vitreoretinal surgical procedures when the surgeon uses an ophthalmoscopic surgical contact lens in surgery. The improvements are achieved by anchoring the ophthalmoscopic surgical contact lens to the eye with the dissolvable medical device to improve stability of the surgical contact lens due to the sticky nature of the dissolvable medical device (corneal shield). The ophthalmoscopic surgical contact lens comprises an optic including an anterior surface having an aspheric base profile and a posterior surface having a shape substantially corresponding to a shape of a cornea of an eye; and a rim comprising a cylindrical tube that circumferentially surrounds the optic and extend above the anterior surface of the optic. The ophthalmoscopic surgical contact lens further comprises a flange. The flange surrounding the optic and the flange has a curvature substantially corresponding to the curvature of a sclera of an eye. The flange further comprises a plurality of tabs extending from a periphery of the flange, wherein each tab is shaped and configured to conform to the curvature of a sclera of an eye.

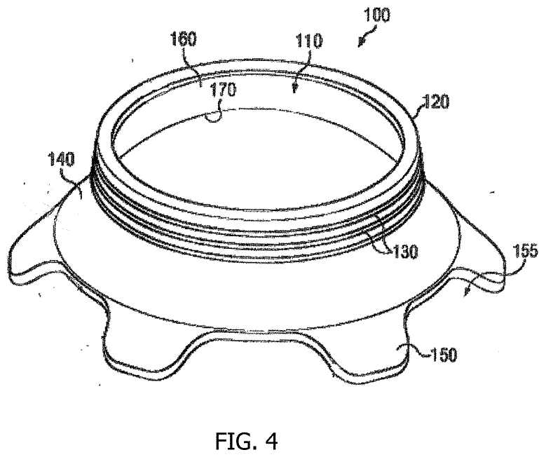

FIG. 4 illustrates an ophthalmoscopic surgical contact lens 100 according to one embodiment of the present application. Though the ophthalmoscopic surgical contact lens 100 shown in FIG. 4 is configured for use in ophthalmologic surgeries, such as vitreoretinal surgery, the contact lens may be used in any ophthalmological context, including diagnosis, treatment, ex vivo evaluation, and postmortem evaluation. The contact lens 100 may comprise a direct ophthalmoscopy lens, for example, of the plano-concave, convex-concave (meniscus), or bi-concave type, or alternatively may be part of a multi-element indirect ophthalmoscopy lens. The contact lens 100 may also be capable of providing irrigation during an ophthalmological procedure. Some embodiments of the contact lens 100 may be configured as disposable single-use ophthalmoscopic surgical lenses, thereby facilitating optimum optics through a new ophthalmoscopic surgical contact lens for each patient.

The ophthalmoscopic surgical contact lens embodiments disclosed herein may be used in combination with a surgical microscope to view the interior of an eye. Such a surgical microscope may be spaced from and cooperate with an embodiment of the surgical contact lens of the present application for capturing light rays exiting the eye through the cornea and passing through the ophthalmoscopic surgical contact lens. The surgical microscope can focus such light rays to create an image of, for example, the retina and the vitreous body.

In the pictured embodiment, the ophthalmoscopic surgical contact lens 100 comprises a one-piece device including integrally formed components. The lens 100 includes a central lens portion or optic 110 circumferentially surrounded by and integrally formed with a cylindrical rim 120, which includes gripping features 130. A circular flange 140, which is integrally formed with the rim 120, extends from and angles away from the rim 120, and a plurality of tabs 150 project outward from the flange 140. A recess 155 is located between any two tabs 150.

The optic 110 is shaped and configured for viewing interior regions of the eye. In some embodiments, the optic 110 may be sized to have an active diameter of approximately 10 mm, which is larger than a typical dilated pupil, to provide adequate light through the optic 110 while remaining small enough to limit interference with a surgeon's hand during an ophthalmological procedure.

As shown in FIG. 4, the optic 110 includes an aspheric anterior optic surface 160 and a posterior optic surface 170 having a curved spherical shape substantially corresponding to the shape of an average human cornea. The aspheric shape of the anterior optic surface 160 allows for enhanced visualization throughout the field of view in comparison to traditional lens geometry by better compensating for, by way of non-limiting example, off-axis stereo viewing, defocus, loss of contrast, and loss of peripheral sharpness.

The invention, in still another aspect, provides a method for conducting ophthalmic surgery in a human eye having a cornea, an anterior chamber, a posterior chamber and a capsular bag located within the posterior chamber, comprising the steps of:

placing a dissolvable medical device for protecting a corneal surface and providing lubricant and hydration to the corneal during ophthalmic surgery, wherein the dissolvable medical device comprising: a polymeric film has sufficient dimensions to substantially cover a cornea when applied to an eye, wherein the polymeric film comprising one or more mucoadhesive polymers, wherein the polymeric film dissolves between 15 minutes to 120 minutes to release the mucoadhesive polymers after applying the polymeric film to the eye, wherein the polymeric film provides more effective corneal surface protection relative to a saline treatment based on Mean Green Fluorescence Index test (demonstrates corneal damage) during a simulated surgery,

surgically opening the human eye, wherein surgically opening is selected from a group consisting of incising, slicing, and injecting the corneal surface.

The method for conducting ophthalmic surgery further comprises a step of placing a second dissolvable medical device after completing ophthalmic surgery for promoting healing of corneal wound.

The following non-limiting Examples are provided to illustrate embodiments of the invention.

### EXAMPLES

Procedure below on how to manufacture and cast corneal shields. Slight variations in volume casted and drying times based on corneal shield thickness (I to III). Target thickness is ˜150 microns).

### Example I

Procedure to make 90-120 micron thickness dry films
Part 1: Procedure to prepare 850 g stock solution of the formulation (HA 40%/HPGuar 40%/PVP 10%/PEG 10%) at 0.85 g/100 mL concentration to make dry films of 90-120 micron thickness:
850 mL of distilled water were placed in a 1 L Erlenmeyer flask followed by the addition of Hyaluronic acid and PVP. The flask was placed in a sonicator, and an overhead mechanical stirrer was set up. The mixture was sonicated and stirrer until a viscous, clear, and homogeneous solution was obtained (90±30 minutes). The speed of the mechanical stirrer was adjusted to 450±50 rpm. HPGuar was added and the mixture was sonicated and stirred for another 90±30 minutes. To the clear, viscous, and homogeneous solution, PEG 400 was added. The mixture was sonicated and stirred for 30 minutes. The mechanical stirring was then stopped, and the sonication was allowed to continue for an additional 30 minutes to release all bubbles.
Film casting procedure:

For the preparation of the films, a petri dish (150 mm diameter×20 mm height) was filled with 200 g±10 g of the stock solution and placed in the evaporation oven.

The oven is equipped with an exhaust fan to move 110 cfm of air. The temperature inside the oven was controlled at 25±3° C. during the evaporation process.
After 40-48 hours of evaporation, the petri dish was taken out of the oven and placed into a plastic zipped bag overnight. The film was then peeled out and kept in a plastic zipped bag at room temperature.

### Example 2

Procedure to make 140-170 micron thickness dry films
Part 1: Procedure to prepare 800 g stock solution of the formulation (HA 40%/HPGuar 40%/PVP 10%/PEG 10%) at 0.85 g/100 mL concentration in order to make dry films of 140-170 micron thickness:
800 mL of distilled water were placed in a 1 L Erlenmeyer flask followed by the addition of Hyaluronic acid and PVP. The flask was placed in a sonicator, and an overhead mechanical stirrer was set up. The mixture was sonicated and stirrer until a viscous, clear, and homogeneous solution was obtained (90±30 minutes). The speed of the mechanical stirrer was adjusted to 450±50 rpm. HPGuar was added and the mixture was sonicated and stirred for another 90±30 minutes. To the clear, viscous, and homogeneous solution, PEG 400 was added. The mixture was sonicated and stirred for 30 minutes. The mechanical stirring was then stopped, and the sonication was allowed to continue for an additional 30 minutes to release all bubbles.
Film casting procedure:
For the preparation of the films, 1) A 1 L beaker was filled with 500 g±10 g of the stock solution and placed in the evaporation oven to reduce the volume to ½ with magnetic stirring. This step takes two days. 2) A petri dish (150 mm diameter×25 mm height) was filled with 270 g±30 g of the concentrated stock solution and placed in the evaporation oven.

The oven is equipped with an exhaust fan to move 110 cfm of air. The temperature inside the oven was controlled at 25±3° C. during the evaporation process.

After 2-3 days of evaporation, the petri dish was taken out of the oven and placed into a plastic zipped bag overnight. The film was then peeled out and kept in a plastic zipped bag at room temperature.

### Example 3

Procedure to make 180-230 micron thickness dry films
Part 1: Procedure to prepare 800 g stock solution of the formulation (HA 40%/HPGuar 40%/PVP 10%/PEG 10%) at 0.85 g/100 mL concentration to make dry films of 180-230 micron thickness:
800 mL of distilled water were placed in a 1 L Erlenmeyer flask followed by the addition of Hyaluronic acid and PVP. The flask was placed in a sonicator, and an overhead mechanical stirrer was set up. The mixture was sonicated and stirrer until a viscous, clear, and homogeneous solution was obtained (90±30 minutes). The speed of the mechanical stirrer was adjusted to 450±50 rpm. HPGuar was added and the mixture was sonicated and stirred for another 90±30 minutes. To the clear, viscous, and homogeneous solution, PEG 400 was added. The mixture was sonicated and stirred for 30 minutes. The mechanical stirring was then stopped, and the sonication was allowed to continue for an additional 30 minutes to release all bubbles.
Film casting procedure:

For the preparation of the films, 1) A 1 L beaker was filled with 750 g±20 g of the stock solution and placed in the evaporation oven to reduce the volume to ½ with magnetic stirring. This step takes two-three days. 2) A petri dish (150 mm diameter×25 mm height) was filled with 300 g±30 g of the concentrated stock solution and placed in the evaporation oven.

The oven is equipped with an exhaust fan to move 110 cfm of air. The temperature inside the oven was controlled at 25±3° C. during the evaporation process.

After 3-4 days of evaporation, the petri dish was taken out of the oven and placed into a plastic zipped bag overnight. The film was then peeled out and kept in a plastic zipped bag at room temperature.

### Example 4

All treatments were given under Isoflurane Sedation

For the eye drop groups (e.g., saline), 1 drop of treatment solution is applied to the assigned eye at the determined frequency

For the dissolvable medical device (Shield) group, a 0.1-0.2 g shield was cut from the sheet and placed on the eye after wetting with PBS and waited until it is secure and dissolved (˜10 mins)

Time under anesthesia and light was aimed to be equal across all groups

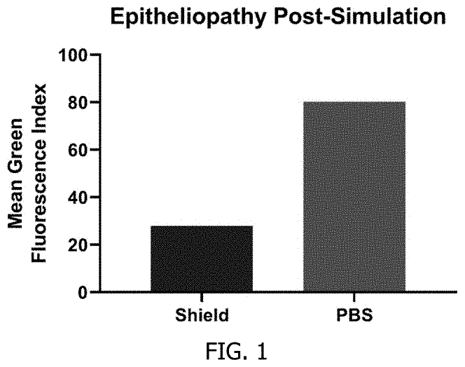

After the dissolvable medical device (Shield) dissolved, mouse was placed under direct light to both eyes to simulate an ocular surgical scenario to both eyes for 1 hour. Right Eye had received the shield while the Left Eye had no shield and was given PBS irrigation every 3-5 minutes regularly (and when taking pictures of the dissolving shield)
FIG. 1 illustrates that the dissolvable medical device provides much more effective protection and does not require regular saline treatment.
Mean Green Fluorescence Index test is provided as follow:
Under Isoflurane Sedation, 1 drop of Fluorescein is placed on the eye to be assessed
After 1 minute, the fluorescein is flushed with PBS
The eye is then placed under blue light and a picture is taken
Settings were unchanged throughout the experiment
The picture is then analyzed through measuring the Mean Green Fluorescence in the area of interest (Cornea)
Mean Green Fluorescein provides an objective measure of corneal epitheliopathy
Epitheliopathy is an inflammatory response. Post-simulation refers to the response after a surgery simulation.

### Example 5

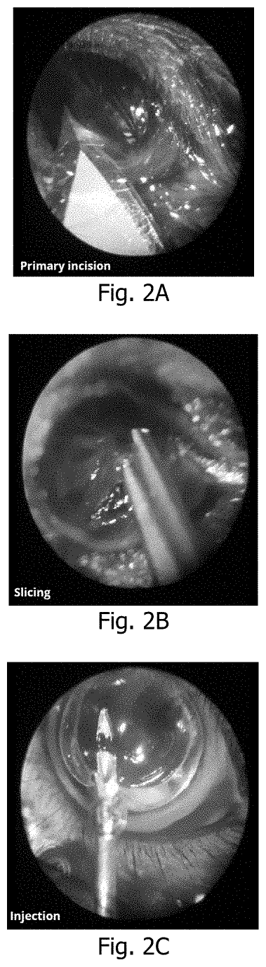

To determine whether the polymeric film causes mechanical impediment to standard surgical procedures. The shield was applied on the eye of an anesthetized mouse and allowed to dissolve for 5 minutes. After that, the mouse was euthanized, and 3 standard ocular surgical techniques were applied. The first was a corneal incision mimicking that of the clean corneal incisions used in cataract surgery. Then, a small needle was inserted in the incision and fluid was injected in simulating the injection of viscoelastic in multiple ocular surgeries. Lastly, a micro-scissor was used to cut around the cornea using different incisions. During all 3 procedures, no added resistance was noted.
FIGS. 2A, 2B and 2C illustrates that the polymeric film does not negatively impact surgical procedure such as making incisions, injections and/or slicing of the cornea.

### Example 6

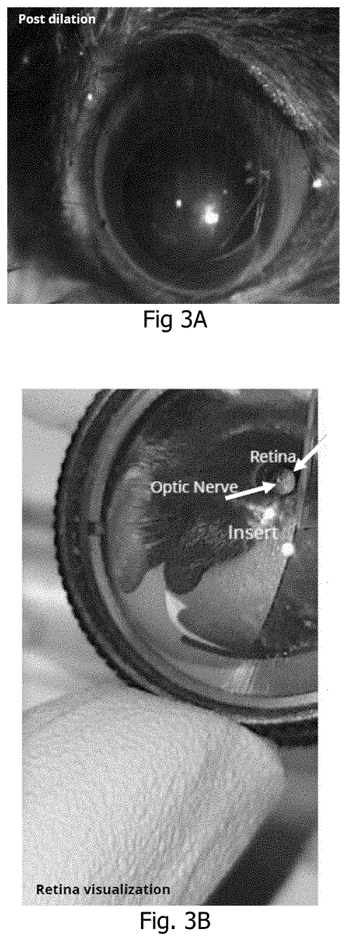

To test whether the shield impedes visualization of the posterior eye, a shield was applied on a euthanized mouse and allowed to dissolve for 5 minutes. After that, a 90D lens was used to visualize the posterior eye. This allowed seeing into the eye and visualizing the optic nerve and the retina.
FIGS. 3A and 3B illustrates that the polymeric film does not impair visualization of retina/retinal hallmarks.

1. A dissolvable medical device for protecting a corneal surface and providing lubrication and hydration to the corneal surface during ophthalmic surgery comprising: a polymeric film has sufficient dimensions to substantially cover a cornea when applied to an eye, wherein the polymeric film comprising one or more mucoadhesive polymers, wherein the polymeric film dissolves between 15 minutes to 120 minutes to release the mucoadhesive polymers after applying the polymeric film to the eye, wherein the polymeric film provides more effective corneal surface protection relative to a saline treatment based on Mean Green Fluorescence Index test (demonstrates corneal damage) during a simulated surgery.

2. The dissolvable medical device of claim 1, wherein the one or more mucoadhesive polymers are selected from the group consisting of: hyaluronic acid or salts thereof, hydroxypropylmethylcellulose (HPMC), methylcellulose, tamarind seed polysaccharide (TSP), guar, hydroxypropyl guar (HP guar), scleroglucan poloxamer, poly(galacturonic) acid, sodium alginate, pectin, xanthan gum, xyloglucan gum, chitosan, sodium carboxymethylcellulose, polyvinyl alcohol, polyvinyl pyrrolidine, carbomer, polyacrylic acid and combinations thereof.

3. The dissolvable medical device of claim 1 wherein the one or more mucoadhesive polymers are HP guar, hyaluronic acid, or sodium hyaluronate or polyvinyl pyrrolidine.

4. The dissolvable medical device of claim 3, wherein the one or more mucoadhesive polymers are present in an amount of at least 5% w/w HP guar, at least 5% w/w hyaluronic acid, at least 5% w/w polyvinyl pyrrolidine by dry weight of the polymeric film and a total amount of mucoadhesive polymers is equal to or less than 100% w/w by dry weight of the dissolvable medical device.

5. The dissolvable medical device of claim 3 further comprising a plasticizer or softener.

6. The dissolvable medical device of claim 5 wherein the plasticizer or softener is selected from the group consisting of: polyethylene glycol (PEG), a PEG derivative, water, Vitamin E, and triethyl citrate.

7. The dissolvable medical device claims 5, wherein the plasticizer or softener is present in an amount of from about 2% to about 30% w/w, about 5% to about 25% w/w, about 5% to about 20% w/w, or about 5% to about 15% w/w of the polymeric film by dry weight of the polymeric film and a total amount of mucoadhesive polymers and the plasticizer or softener is equal to or less than 100% w/w by dry weight of the dissolvable medical device.

8. The dissolvable medical device of claim 6, wherein the plasticizer or softener is PEG.

9. The dissolvable medical device of claim 1, wherein the insert is comprised of approximately 40% HP guar, approximately 10% PVP, approximately 40% sodium hyaluronate, and approximately 10% PEG.

10. A kit for use in ophthalmic surgical procedures comprising: 1) at least one dissolvable medical device for protecting a corneal surface and providing lubrication and hydration to the cornea before and during an ophthalmic surgery comprising: a polymeric film has sufficient dimensions to substantially cover a cornea when applied to an eye, wherein the polymeric film comprising one or more mucoadhesive polymers, wherein the polymeric film dissolves between 15 minutes to 120 minutes to release the mucoadhesive polymer after applying the polymeric film to the eye, wherein the polymeric film provides more effective corneal surface protection relative to a saline treatment based on Mean Green Fluorescence Index test (demonstrates corneal damage) during a simulated surgery, and 2) at least one viscosurgical viscoelastic or at least one ophthalmoscopic surgical contact lens, wherein the at least one viscosurgical viscoelastic comprising a cohesive viscoelastic, wherein cohesive viscoelastic is a hyaluronate-based viscoelastic, wherein the at least one ophthalmoscopic surgical contact lens comprising: an optic including an anterior surface having an aspheric base profile and a posterior surface having a shape substantially corresponding to a shape of a cornea of an eye; and a rim comprising a cylindrical tube that circumferentially surrounds the optic and extend above the anterior surface of the optic.

11. The kit for use in ophthalmic surgical procedures of claim 10, further comprising a dispersive viscoelastic, wherein the dispersive viscoelastic is a combination of hyaluronic acid and chondroitin sulfate, or ophthalmically acceptable salts thereof, in an ophthalmically acceptable vehicle.

12. The kit for use in ophthalmic surgical procedures of claim 10, comprising two dissolvable medical devices, wherein the two dissolvable medical devices are same or different.

13. The kit for use in ophthalmic surgical procedures of claim 12, wherein one dissolvable medical device is applied to a corneal surface before or during ophthalmic surgery for protecting a corneal surface and providing lubricant and hydration and the other one dissolvable medical device is applied to a corneal surface after ophthalmic surgery for promoting healing of corneal wound.

14. The kit for use in ophthalmic surgical procedures of claim 10, wherein the rim comprises a gripping feature.

15. The kit for use in ophthalmic surgical procedures of claim 10, wherein the at least one ophthalmoscopic surgical contact lens further comprising a flange, wherein the flange surrounding the optic, wherein the flange has a curvature substantially corresponding to the curvature of a sclera of an eye.

16. The kit for use in ophthalmic surgical procedures of claim 14, wherein the flange further comprising a plurality of tabs extending from a periphery of the flange, wherein each tab is shaped and configured to conform to the curvature of a sclera of an eye.

17. A method for conducting ophthalmic surgery in a human eye having a cornea, an anterior chamber, a posterior chamber, and a capsular bag located within the posterior chamber, comprising the steps of:
placing a first dissolvable medical device for protecting a corneal surface and providing lubricant and hydration to the corneal during ophthalmic surgery, wherein the dissolvable medical device comprising: a polymeric film has sufficient dimensions to substantially cover a cornea when applied to an eye, wherein the polymeric film comprising one or more mucoadhesive polymers, wherein the polymeric film dissolves between 15 minutes to 120 minutes to release the mucoadhesive polymers after applying the polymeric film to the eye, wherein the polymeric film provides more effective corneal surface protection by at least 100% comparing to a saline treatment based on Mean Green Fluorescence Index test (demonstrates corneal damage) during a simulated surgery;
surgically opening the human eye, wherein surgically opening is selected from a group consisting of incising, slicing, and injecting the corneal surface.

18. A method for conducting ophthalmic surgery of claim 14, further comprising a step of placing a second dissolvable medical device after completing the ophthalmic surgery.
